(19) United States
(12) Patent Application Publication
(10) Pub. No.: US 2020/0060808 A1
(43) Pub. Date: Feb. 27, 2020

(54) DEVICE TO FACILITATE PERFORMING DESCEMET'S MEMBRANE ENDOTHELIAL KERATOPLASTY (DMEK)

(71) Applicant: THE JOHNS HOPKINS UNIVERSITY, Baltimore, MD (US)

(72) Inventors: Eric Chiang, Baltimore, MD (US); Kali Barnes, Baltimore, MD (US); Conan Chen, Baltimore, MD (US); Anshul Subramanya, Baltimore, MD (US); Stephanie Cai, Baltimore, MD (US); Allen O. Eghrari, Baltimore, MD (US)

(21) Appl. No.: 16/612,035

(22) PCT Filed: May 8, 2018
(86) PCT No.: PCT/US2018/031521

§ 371 Date: Nov. 8, 2019

Related U.S. Application Data
(60) Provisional application No. 62/502,839, filed on May 8, 2017.

(51) Int. Cl. A61F 2/14; A61F 9/007

(52) U.S. Cl. CPC A61F 2/142; A61F 2/148; A61F 9/007

(57) ABSTRACT

An embodiment in accordance with the present invention provides a device and method for performing Descemet's membrane endothelial keratoplasty (DMEK). The device includes a tray for loading a corneal graft, a permeable cap, and a handle for facilitating delivery of the corneal graft into the eye of the recipient. The tray is configured such that the corneal graft is tri-folded on the tray or folded on the donor cornea. The permeable cap allows for hydration and protection of the graft during transit. When it is time to deliver the corneal graft into the eye of the recipient, the tray can be loaded onto the handle after the caps are be removed. A method according to the present invention includes a corneal graft being loaded onto the tray, covered with the permeable cap, and transmitted to the facility doing the corneal transplant.

### CROSS REFERENCE TO RELATED APPLICATIONS

This application claims the benefit of U.S. Provisional Patent Application No. 62/502,839 filed on May 8, 2017, which is incorporated by reference, herein, in its entirety.

### FIELD OF THE INVENTION

The present invention relates generally to medical devices. More particularly, the present invention relates to a device for facilitating Descemet's membrane endothelial keratoplasty (DMEK) procedures.

### BACKGROUND OF THE INVENTION

Over 3 million patients, representing 4% of Americans over the age 40, suffer from severe eye pain and possible blindness due to diseased corneas. Corneal diseases like Fuchs dystrophy and pseudophakic bullous keratopathy (PBK), induce severe eye pain, decreased visual acuity, and eventual blindness due to fluid accumulation in the cornea. There are two primary surgeries for treatment: Descemet's stripping automated endothelial keratoplasty (DSAEK) and Descemet's membrane endothelial keratoplasty (DMEK). In DSAEK a thick layer of tissue of 50 to 120 microns is transplanted, and in DMEK a layer of 10 to 15 microns thick is transplanted. DSAEK is the more established procedure, constituting 89% of all EKs in 2014, compared to 11% for DMEK. However, DMEK outperforms DSAEK by nearly every post-operative metric, including shorter recovery times and better restoration of visual acuity, but surgeons are still hesitant to adopt DMEK over DSAEK. This hesitancy stems from the higher difficulty of DMEK compared to its counterpart. A DMEK tissue graft is significantly thinner than DSAEK tissue, and the lack of specialized tools to handle this graft deters surgeons from adopting DMEK. The DMEK paradigm can be best described by the words of Dr. Katelyn Earls of the Johns Hopkins Wilmer Eye Institute: “If DMEK were easier, everyone would do it.”

In a representative DMEK procedure, eye banks receive entire corneas that have been donated for surgeries. Next, eye bank technicians harvest and prepare the DMEK graft, a single layer of corneal tissue, by peeling a circular portion of this layer off of the donor cornea. FIG. 1 illustrates the layers of corneal tissue. The prepared donor cornea, with the graft still partially attached, is then shipped to the hospital. Eye banks are the sole source of grafts for hospitals. In the operating room, the surgeon begins the DMEK by first peeling the entire graft off of the prepared donor cornea. Due to its tissue properties, the graft scrolls tightly upon itself, as illustrated in FIG. 2, similar to how wrapping paper scrolls up when pulled off the roll, and any physical contact with the DMEK scroll causes immediate cell death. FIG. 2 illustrates a scrolled DMEK graft after insertion into the eye. The surgeon loads the scrolled graft into his or her device of choice and inserts the graft into the patient's eye. Finally, the graft is unrolled into a flat disc by tapping on the top of the cornea and shooting jets of fluid inside the patient's eye, as illustrated in FIG. 2. Special care must be taken to minimize direct physical contact with the graft to avoid damage to the corneal graft.

DMEK is quickly gaining traction in the endothelial keratoplasty (EK) community; from 2013 to 2015, the number of DMEKs increased 208%. This explosive growth over the past few years can be attributed to its ability to surpass DSAEK in nearly every postoperative measure. Patients who undergo DMEK are able to restore their vision to their pre-diseased levels, with studies reporting maximum restored visual acuity of 20/20, whereas the highest level for DSAEK was 20/40. This implies that there are patients who are undergoing DSAEK, when they should be undergoing DMEK.

Additionally, DMEK requires shorter recovery times of 1 to 3 months, rather than the 6 to 12 months required for DSAEK. Finally, studies indicate that DMEK has a lower graft rejection rate than DSAEK, at an average of 2% as opposed to 4%. In spite of these results, many corneal surgeons are still hesitant to perform DMEK over DSAEK. The greatest hindrance to widespread DMEK adoption is the difficulty surgeons have with handling the graft in the operating room. Much of this difficulty stems from the thinness and fragility of the grafts used in DMEK. FIG. 3A illustrates an eye bank technician partially peeling graft. FIG. 3B illustrates a surgeon fully peeling graft. FIG. 3C illustrates a DMEK graft stained and scrolled in BSS. FIG. 3D illustrates a graft being loaded into standard injector. FIG. 3E illustrates a graft being injected into patient eye. FIG. 3F illustrates a surgeon attempting to unroll scrolled graft by shooting fluid.

In further detail, FIGS. 3A-3F illustrate a timeline of the graft's journey from the eye tissue bank, where it is prepared, to the operating room, where it is transplanted, is as follows:

1. An eye bank receives a cornea from an organ donor and a trained technician at the eye bank prepares the DMEK graft by separating a single layer of endothelial cells from the donor cornea (FIG. 3A).

2. The graft is left partially attached to the donor cornea according to the specifications of the surgeon, and the cornea is stored in bio-compatible fluid in a transport container.

3. The packaged cornea is shipped to the surgeon.

4. In the operating room, the surgeon fully peels the graft off of the donor cornea (FIG. 3B) and dyes the graft with a blue stain. The graft is submerged in a dish of balanced salt solution (BSS), where it scrolls up tightly (FIG. 3C).

5. The surgeon loads the stained graft into an inserter device (FIG. 3D) and inserts the graft into the patient's eye (FIG. 3E).

6. The surgeon must unscroll the graft in its correct orientation without physically contacting it. The only methods of doing this are by shooting jets of fluid into the eye (FIG. 3F) or tapping on the surface of the eye with a bent cannula.

7. An air bubble is injected under the graft to secure it in place, thus completing the surgery.

There is currently no specialized device for steps 4-7 in the timeline above. The ophthalmologist is limited to makeshift tools that are either assembled in the operating room or shoehorned in from other surgical fields. One of the most common “devices” used for step 5 above is the Bonfadini-Todd injector and is composed of cut IV tubing, a syringe, and a spare cataract cartridge that does not belong in a corneal surgery.

By far the most difficult aspect of the procedure for the surgeon stems from the delicacy and scrolling nature of the DMEK graft. The thinness of the graft causes it to naturally scroll (FIG. 3C), similar to wrapping paper. Any physical contact to the graft causes immediate cell death, and this presents the most problems during step 6. Once the DMEK graft is inserted into the patient eye it must be unscrolled, and the fragility of the graft limits surgeons to shooting fluid at the scroll inside the eye, or physically tapping on the surface of the eye. These primitive and unreliable methods cause the time it takes to unroll the DMEK graft to be highly variable, from one minute to two hours, and the patient is typically awake during this procedure. In contrast, DSAEK requires a consistent 30 minutes per surgery.

It would be ideal if DMEK surgeries could be performed in place of DSAEK whenever possible, but the nuances, difficulty, and unreliability of the DMEK procedure drive surgeons away. Therefore, a need exists to facilitate the DMEK procedure and reduce variability in surgery time, primarily by facilitating the unscrolling of the DMEK graft and limiting DMEK graft cell death. The need is summarized perfectly by Dr. Michael Coleman, from the Johns Hopkins School of Medicine: “If there was anything that could make [DMEK] more predictable, I would use it.”

Therefore, it would be advantageous to provide a device for facilitating DMEK procedures.

### SUMMARY OF THE INVENTION

The foregoing needs are met, to a great extent, by the present invention, wherein in one aspect a device for Descemet's membrane endothelial keratoplasty (DMEK) includes an inserter for holding a corneal graft. The inserter is configured to hold the corneal graft in a tri-folded orientation. The inserter includes a distal end configured for insertion into the eye and a proximal end that is wider than the distal end. The device includes a cap configured to couple to the inserter. The cap is configured for covering the corneal graft in the inserter. The device also includes a handle that is configured to couple to the inserter for facilitating delivery of the corneal graft to an eye of a patient.

In accordance with an aspect of the present invention, the inserter includes a distal end with a flattened, ovular shape. The cap can have a front component and a back component that interlock. The handle can have a straight configuration, or in other embodiments the handle can be bent at an angle to the inserter. The handle is configured for facilitating a pull through delivery of the corneal graft to an eye of a patient. The handle can also be configured for facilitating a fluid based delivery of the corneal graft to an eye of a patient. The distal end of the inserter can have a circular cross section and a bevel, and the proximal end of the inserter can have an ovular cross-section and protrusions for attachment of modular interlocking handle pieces. The inserter defines a sufficient interior space and exterior protection to allow the graft to be stored in Optisol and in a trifold configuration. The inserter further includes luminal axial troughs running from the proximal end halfway up a length of the inserter. Multiple troughs are arranged radially such that the corneal graft will lie in the tri-folded configuration with minimal luminal wall contact. Arrangement and depth of the troughs also allows for fluid flow and facilitate grasping of graft by forceps. The handle has the capability to aspirate and eject fluid. The cap can take the form of a fluid-permeable cap. Alternately the cap can take the form of a fluid-tight cap.

In accordance with another aspect of the present invention, a method for Descemet's membrane endothelial keratoplasty (DMEK) includes placing a corneal graft on an inserter for holding the corneal graft. The method includes covering the corneal graft with a cap that is configured to couple to the inserter. The method also includes transmitting the corneal graft to the surgical center for performing the DMEK procedure.

In accordance with yet another aspect of the present invention, the method includes storing the corneal graft in the inserter in a biocompatible fluid. The method includes storing the corneal graft in the inserter in Optisol. The method includes storing the corneal graft in a tri-fold configuration. The method includes folding the graft in an opposite direction of a direction in which it naturally scrolls. The method also includes packaging the inserter for shipment.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates the layers of corneal tissue.

FIG. 2 illustrates a scrolled DMEK graft after insertion into the eye.

FIGS. 3A-3F illustrate a timeline of the graft's journey from the eye tissue bank, where it is prepared, to the operating room, where it is transplanted.

FIG. 4A illustrates a flow chart of the current workflow for contrast.

FIG. 4B illustrates a flow chart of a method of the present invention.

FIGS. 5A-5C illustrate the tri-folded graft being folded in a tray of the device and unfurling as it is being removed from the device.

FIG. 6 illustrates a perspective view of a tray of the device, according to an embodiment of the present invention.

FIG. 7 illustrates a tray of the present invention storing a dyed graft, according to an embodiment of the present invention.

FIGS. 8A-8B illustrate perspective views of an inserter according to an embodiment of the present invention.

FIGS. 9A-9C illustrate perspective views of the single piece cap, according to an embodiment of the present invention.

FIGS. 10A-10D illustrates perspective views of the steps to cover the device with the cap, according to an embodiment of the present invention.

FIG. 11A illustrates a front view of a front cap. FIG. 11B illustrates a top down view of a front cap. FIG. 11C illustrates a right-side view of a front cap.

FIG. 12A illustrates a front view of a back cap, FIG. 12B illustrates a right-side view of a back cap, and FIG. 12C illustrates a top-down view of a back cap.

FIGS. 13A-13C illustrate views of a straight handle according to an embodiment of the present invention.

FIGS. 14A-14C illustrate views of a straight handle according to an embodiment of the present invention.

FIGS. 15A-15C illustrate perspective views of an inserter of the device according to an embodiment of the present invention.

FIG. 16 illustrates a perspective view of a cap, according to an embodiment of the present invention.

FIGS. 17A-17B illustrate perspective views of a handle according to an embodiment of the present invention.

FIGS. 18A-18E illustrate perspective views of another embodiment of the inserter, according to the present invention.

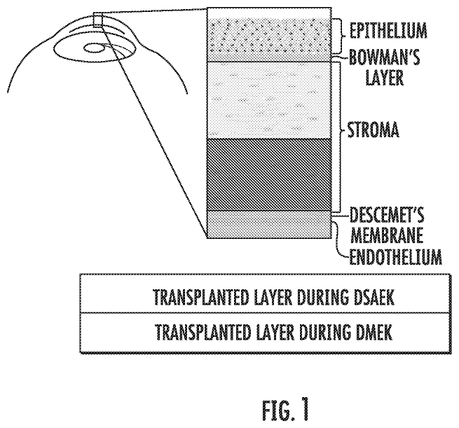

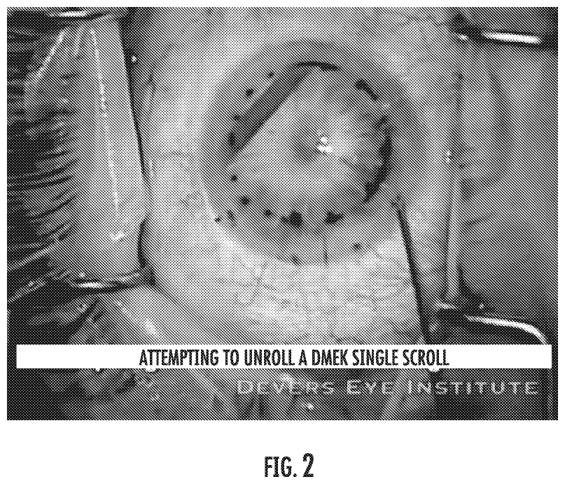

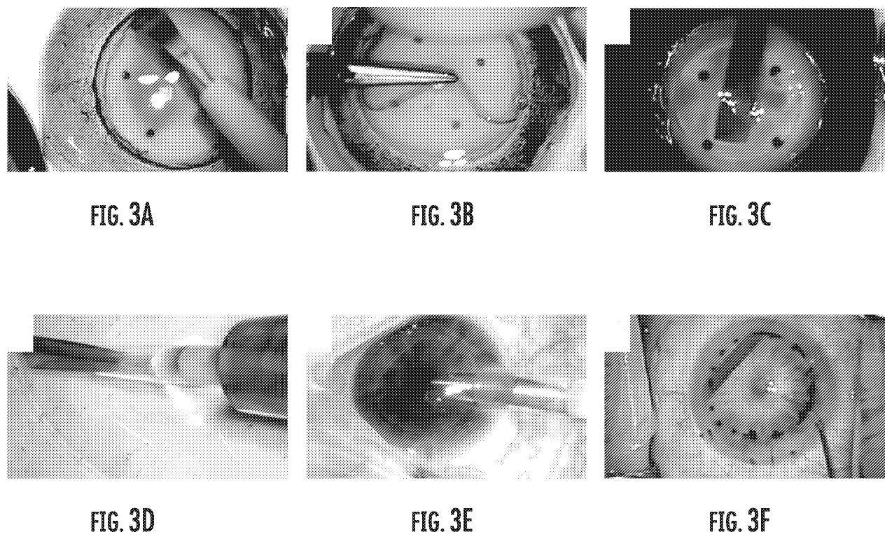

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device and method for performing Descemet's membrane endothelial keratoplasty (DMEK). The device includes a tray for loading a corneal graft, a permeable cap, and a handle for facilitating delivery of the corneal graft into the eye of the recipient. The tray is configured such that the corneal graft is tri-folded on the tray. The corneal graft can also be folded on the donor cornea. The permeable cap allows for hydration and protection of the graft during transit. When it is time to deliver the corneal graft into the eye of the recipient, the tray can be loaded onto the handle and the caps can be removed. Alternately, the caps are removed first, and then the tray is loaded onto the handle. A method according to the present invention includes a corneal graft being loaded onto the tray, covered with the permeable cap, and transmitted to the facility doing the corneal transplant.

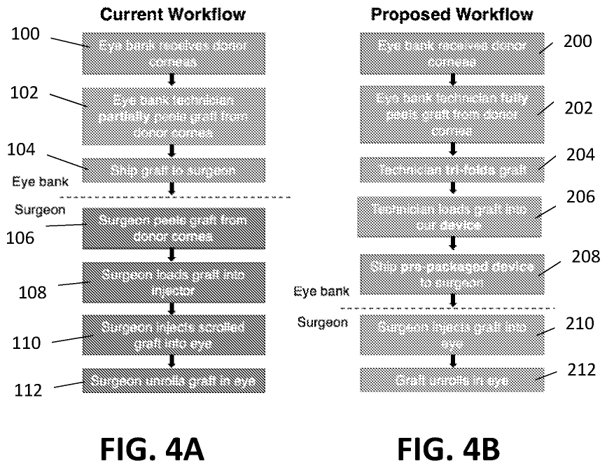

FIG. 4A illustrates a flow chart of the current workflow used at eye banks. In contrast, the device of the present invention eliminates the difficult aspects of the DMEK procedure for surgeons by modifying the surgical workflow and reallocating risk to skilled eye bank technicians. FIG. 4B illustrates a flow chart of a method of the present invention. As illustrated in FIG. 4B, the method of the present invention provides clinicians with fully prepared DMEK grafts that have been pre-loaded into the device of the present invention. With respect to FIG. 4A, currently, donor corneas are received at eye banks in step 100. Skilled technicians separate or peel a small portion of the DMEK graft from the donor cornea in step 102 and send the cornea to the hospital in step 104, leaving surgeons with the high risk and difficult steps of preparing and manipulating the graft. In step 106, the surgeon peels the graft from the donor cornea. The surgeon then loads the graft into the injector in step 108, and injects the scrolled graft into the eye in step 110. Finally, the surgeon unrolls the graft in the eye 112.

In contrast, according to the present invention, illustrated in FIG. 4B, the eye bank receives the cornea in step 200, but skilled eye bank technicians instead fully separate the graft from the donor cornea in step 202. The skilled technician “tri-folds” the entire donor graft in step 204, a technique that folds the graft opposite the direction in which it naturally scrolls, similar to folding wrapping paper with the design facing inwards. Just like how wrapping paper will unfold itself when left alone, so too will the DMEK graft when inserted into the eye. The tri-folded graft is loaded into the device of the present invention in step 206, which is then shipped directly to hospitals or surgical centers in step 208. In the operating room, the surgeon need only un-package the device of the present invention, inject the graft in step 210, and watch as the graft properly orients itself, and unfolds in the correct orientation in step 212.

A device according to the present invention is designed to complement all aspects of this optimized workflow. The device includes a wide loading tray onto which eye bank technicians can easily transfer the donor graft and perform the tri-folding step. Once the graft is loaded, a permeable cap is placed over the platform. In a preferred embodiment of the present invention, the permeable cap includes a first cap for one end of the tray and a second for the other end of the tray to both hydrate and secure the graft during transport. Finally, the device of the present invention utilizes a flattened front tip in contrast to round tips currently found on the market. This ensures the graft does not prematurely unfold while loaded. The flattened front tip configuration also reduces stress on the patient's eye during the injection step. These features along with the workflow offload risk from the surgeon, standardize the procedure, and greatly reduce the risk of graft failure.

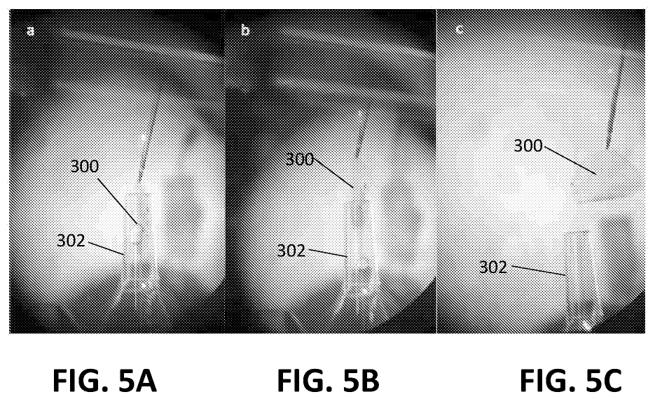

The device of the present invention is the first to utilize the tri-fold technique, which is a proven time-saving and reliable method for performing DMEK. FIGS. 5A-5C illustrate the tri-folded graft being loaded into the tip of the device and unfurling as it is being removed from the device. All high-risk preparation steps are offloaded to eye bank technicians, who are more skilled in manipulating DMEK grafts than surgeons. The DMEK graft is pre-packaged into the device of the present invention, which will allow the surgeon to circumvent risky steps of the procedure and thus standardize operation times. FIG. 5A illustrates a graft 300 being inserted into a tip 302 of a device according to an embodiment of the present invention. This action is completed by the skilled eye bank technician. FIGS. 5B and 5C illustrate the graft 300 being removed from the tip of the device to show how the graft 300 unfurls as it is released from the device.

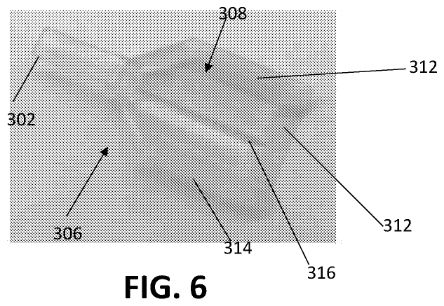

The device of the present invention takes the form of a novel cornea transplant inserter to be used during DMEK, as illustrated in FIG. 6. FIG. 6 illustrates a perspective view of an inserter 306 of the device, according to an embodiment of the present invention. The device of the present invention takes advantage of the natural scrolling tendency of DMEK tissue rather than working against it. The graft will be tri-folded and packaged into a device in a formation that allows for easy insertion, guaranteed correct tissue orientation, and minimal tissue manipulation. Surgeons will no longer be solely responsible for manipulating and transplanting the graft into the patient's eye and will reap the benefits of an easier procedure and standardized procedure times.

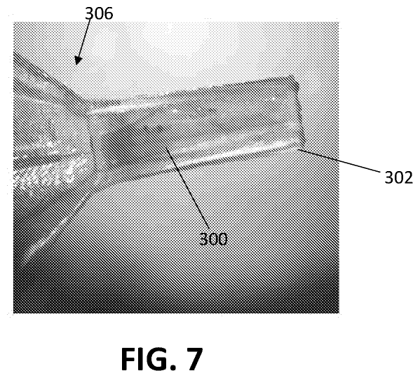

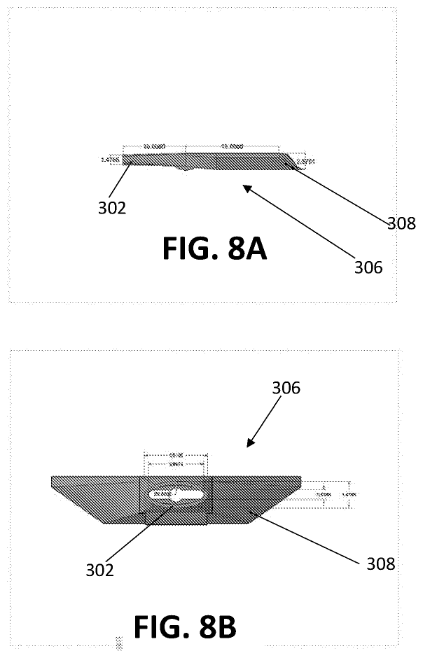

FIG. 7 illustrates a tray of the present invention storing a dyed graft, according to an embodiment of the present invention. The device of the present invention utilizes a specially designed tip 302 to reduce stress on the patient's eye while also maintaining the trifold configuration of the graft 300, as illustrated in FIG. 7. FIGS. 8A-8B illustrate perspective views of an inserter of the device according to an embodiment of the present invention. FIG. 8A illustrates a right-side view of the inserter 306, and FIG. 8B illustrates a front view of the inserter 306. The tip 302 is flattened and ovular; this decreased height reduces the vertical wound strain on the patient's eye when the tip 302 is inserted into the eye, thus decreasing the invasiveness of the procedure. However, the device still fits into a 3.5 millimeter incision, which is standard to the DMEK procedure. A 3.5 millimeter incision is able to naturally heal without sutures, and the small incision is considered a significant advantage of DMEK over DSAEK, which requires a 5-6 mm incision. The flattened tip 302 also ensures that the graft is secured within the device, while it is being transported. During preliminary storage testing, rotational graft movement was minimized due to the flattened geometry of the tip 302. The shape of the tip 302 not only prevents the graft from being damaged in transport, but also standardizes graft delivery for the physician.

The base of the device of the present invention is designed to facilitate all aspects of graft manipulation from the cornea to the tri-fold formation. The base 308 of the device 304, is illustrated in FIG. 6. The base 308 possesses a wide loading platform 310 with a scoop 312 at its base. This allows the eye bank technician to scoop the trifolded graft out of the donor cornea and onto the wide loading platform. Upwards slanted sides 314 keep the tissue in place by preventing it from sliding off the edges, while its curved contour fits the average radius of cornea curvature thus making it appropriate for most tissue.

A 22 gauge groove 316 runs down the center of the device along the wide loading base and the flattened inserter tip. This groove 316 serves two purposes. Firstly, it facilitates fluid transfer between the interior of the tip and its surrounding fluids during transport, which is a key aspect in maintaining graft viability when the loaded device is shipped to the surgeon. Secondly, this groove is designed to allow for easy movement of the tri-folded graft into and out of the inserter tip 302 by providing clearance for 23 gauge microforceps: the current standard of microforceps. Thus, this groove facilitates the usage of the device of the present invention without needing to change the current armamentariums of both the eye bank and the surgeon.

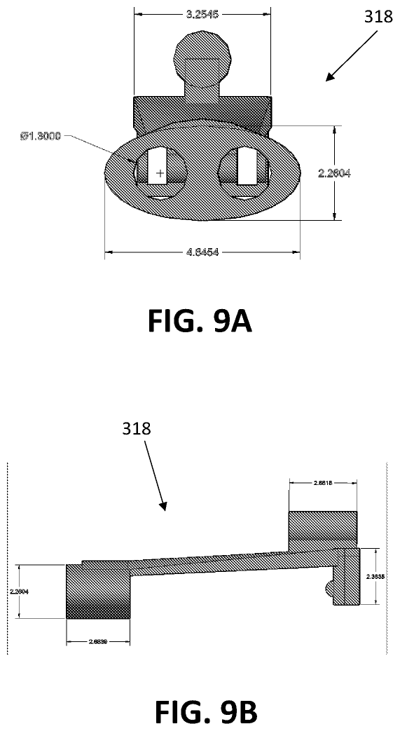

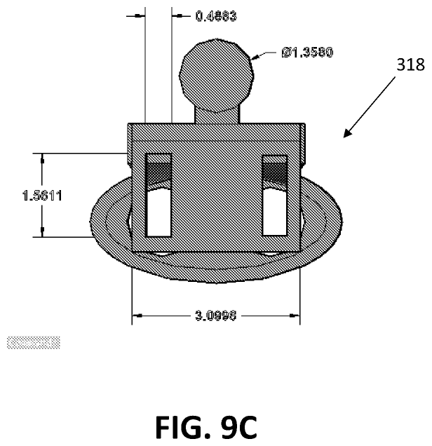

A permeable cap illustrated in FIGS. 9A-9C secures the graft 300 within the device 304 during shipping while allowing the flow of storage medium into and out of the inserter tip 302. Once the graft is loaded within the device, the permeable cap 318 utilizes a snap-lock mechanism 320 that securely fits onto the device but also allows easy removal of the cap 318 by the gloved surgeon in the operating room. The front of the cap has narrow, vertical slits or small holes 322 to promote fluid exchange which, coupled with the medial groove, allow the graft to thrive during transport from eye bank to surgeon.

FIGS. 9A-9C illustrate perspective views of the single piece cap, according to an embodiment of the present invention. FIG. 9A illustrates a front view of a single cap 318. FIG. 9B illustrates a right side view of a single cap 318. FIG. 9C illustrates a rear view of a single cap 318. One variation of the cap (called single-piece cap) slips on to the front of the inserter tip and snaps on to the back of the tip. Another variation of the cap (called double-piece cap) uses two pieces, one piece to cap the front and the other to cap the back. The front cap and the back cap interlock.

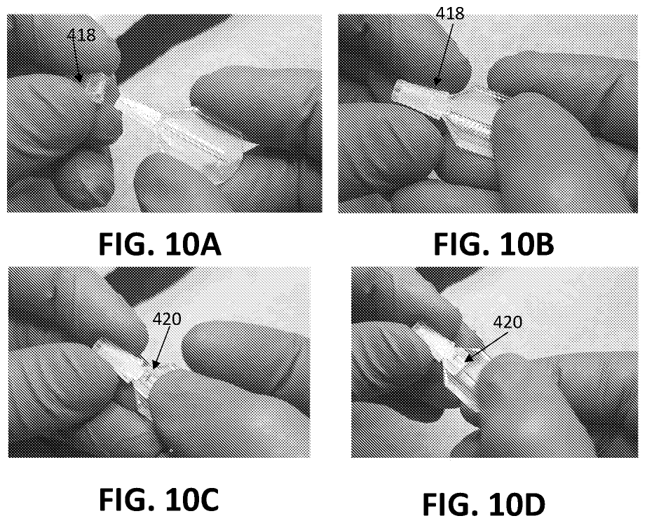

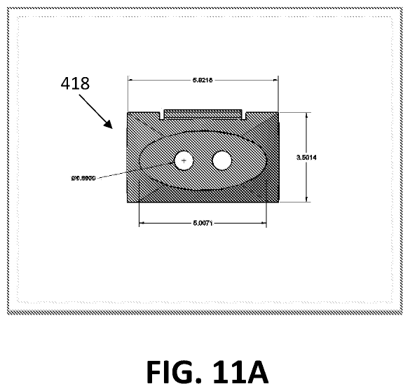

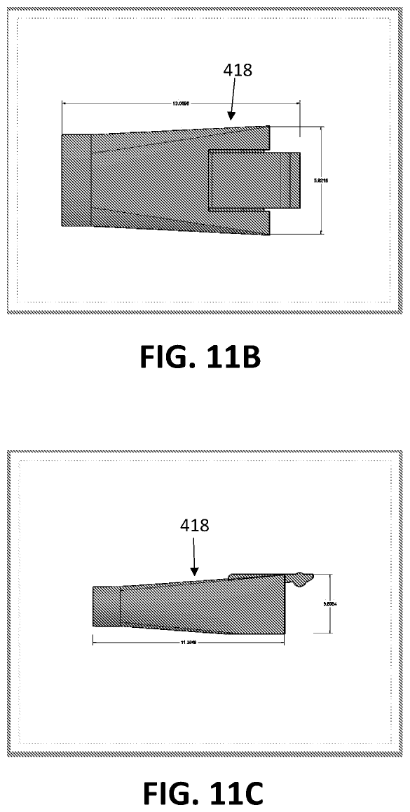

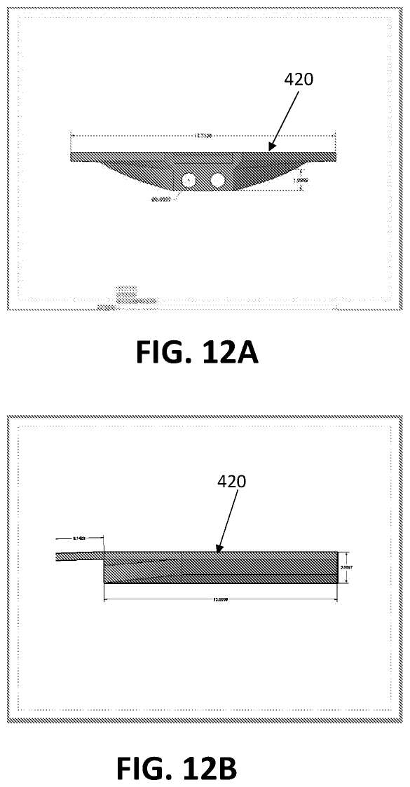

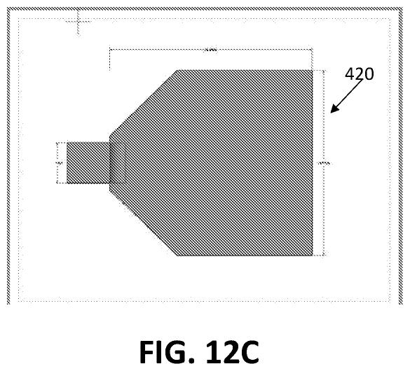

FIGS. 10A-10D illustrates perspective views of the steps to cover the device with the cap, according to an embodiment of the present invention. A two piece cap is illustrated in FIGS. 10A-10D. FIGS. 10A and 10B illustrate placing on the front cap 418, and FIGS. 10C and 10D illustrate placing on the back cap 420, according to an embodiment of the present invention. FIG. 11A illustrates a front view of a front cap 418. FIG. 11B illustrates a top down view of a front cap 418. FIG. 11C illustrates a right-side view of a front cap 418. FIG. 12A illustrates a front view of a back cap 420, FIG. 12B illustrates a right-side view of a back cap 420, and FIG. 12C illustrates a top-down view of a back cap 420.

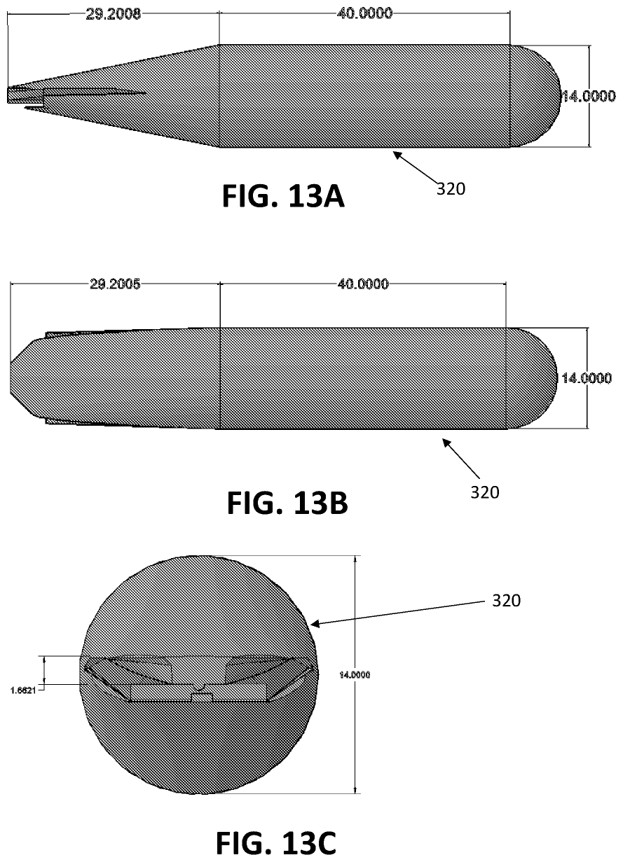

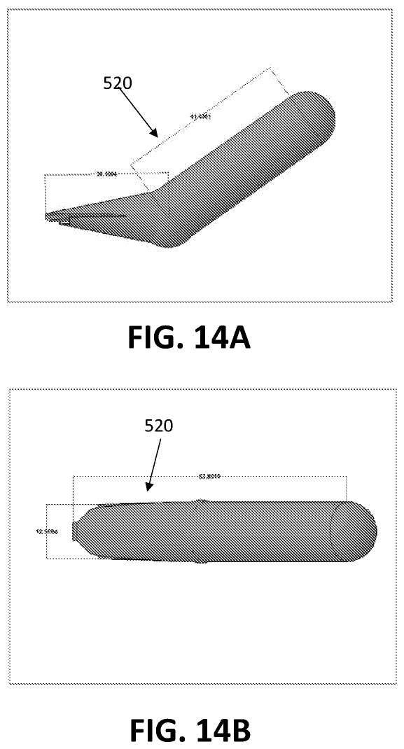

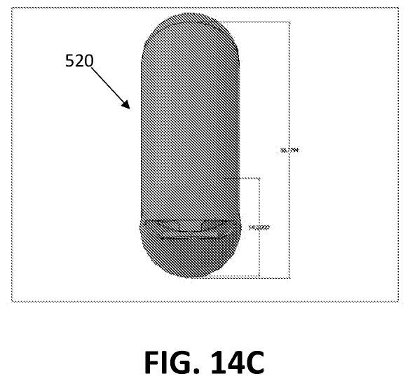

The device of the present invention includes an ergonomic handle 320 that allows surgeons to easily manipulate the device during the surgery and within the corneal space. After the pre-loaded device is delivered to the surgeon, the permeable cap 318 is removed and the handle 320 is fitted into place by the surgeon. Alternately, the handle 320 can be fitted to the device while the permeable cap 318 is still in place. The handle 320 is designed to make the device easy to hold and easy to use. The handle 320 can be straight or can be at a slight angle relative to the inserter 306 holding the corneal graft 300. FIGS. 13A-13C illustrate views of a straight handle according to an embodiment of the present invention. FIG. 13A illustrates a side view of a straight handle 320, FIG. 13B illustrates a top-down view of the straight handle 320, and FIG. 13C illustrates a front-side view of the straight handle 320. FIGS. 14A-14C illustrate views of a bent handle according to an embodiment of the present invention. FIG. 14A illustrates a side view of a bent handle 520, FIG. 14B illustrates a top-down view of the bent handle 520, and FIG. 14C illustrates a front-side view of the bent handle 520.

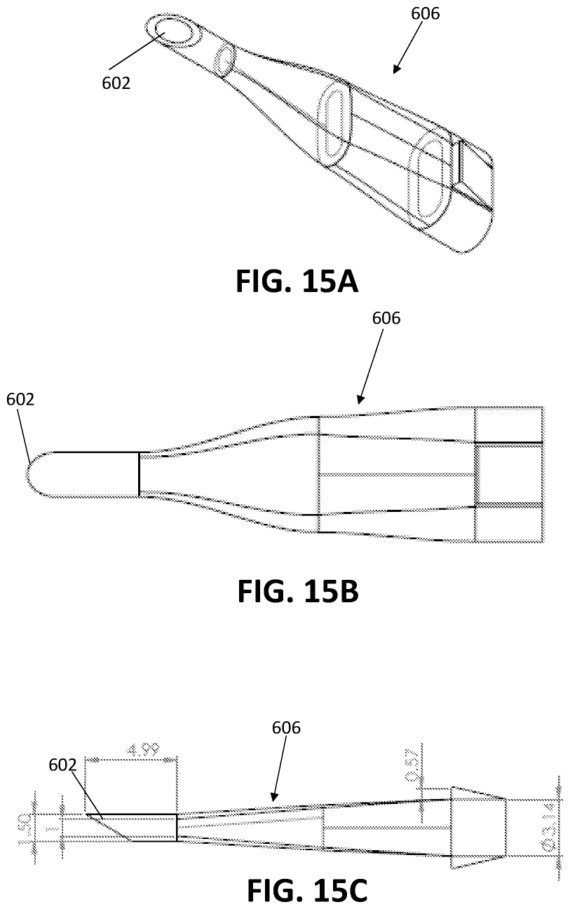

FIGS. 15A-15C illustrate perspective views of an inserter of the device according to an embodiment of the present invention. FIG. 15A illustrates a perspective view of the inserter 606, FIG. 15B illustrates a top-down view of the inserter 606, and FIG. 15C illustrates a side view of the inserter 606. The tip 602 is angled. However, the device still fits into a 3.5 millimeter incision, which is standard to the DMEK procedure.

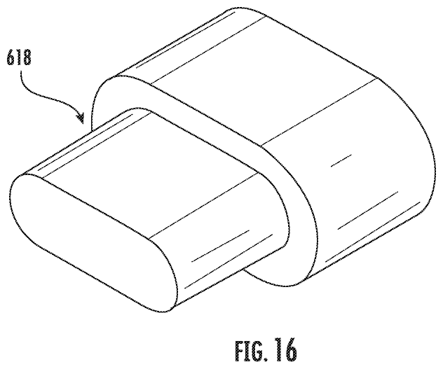

FIG. 16 illustrates a perspective view of a cap, according to an embodiment of the present invention. The cap 618, in some embodiments, can take the form of a water-tight cap 618. The cap 618 is configured for covering the corneal graft in the tube.

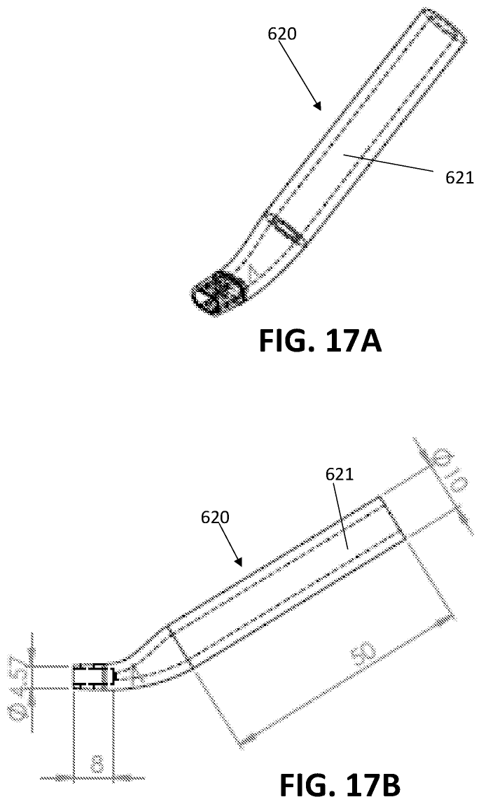

FIGS. 17A-17B illustrate perspective views of a handle according to an embodiment of the present invention. FIG. 17A illustrates a perspective view of the handle 620, and FIG. 17B illustrates a side view of the handle 620. As illustrated in FIGS. 17A and 17B, the handle 620 is bent. The handle 620 can include a lumen 621 to allow for aspiration and ejection of fluid. The handle 620 and the inserter can be configured to snap-fit, frictionally fit, or any other suitable means of coupling the inserter to the handle 620.

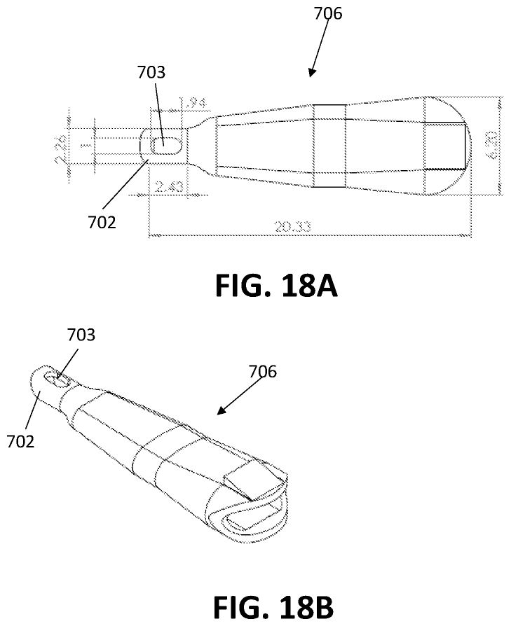

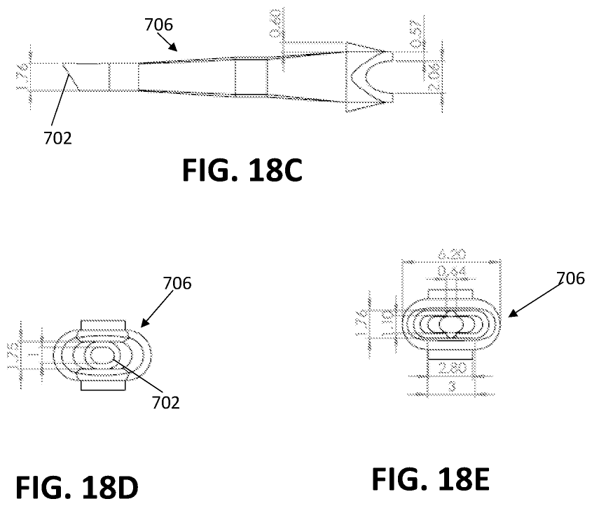

FIGS. 18A-18E illustrate perspective views of another embodiment of the inserter, according to the present invention. FIG. 18A illustrates a top down view of the inserter 706, FIG. 18B illustrates a perspective view of the inserter 706, FIG. 18C illustrates a side view of the inserter 706, FIG. 17D illustrates a front-end view of the inserter 706, and FIG. 18D illustrates a back-end view of the inserter 706. The tip 702 is angled. However, the device still fits into a 3.5 millimeter incision, which is standard to the DMEK procedure. The tip 702 can also include an aperture 703.

Preferably, the device will be made from a transparent, biocompatible material such that the corneal graft can be visualized within the tray. The device can be formed from a plastic or other material that is biocompatible and known to or conceivable to one of skill in the art. The components of the device can be molded, 3D printed, machined, or other method of manufacture known to or conceivable to one of skill in the art. Within the procedure, the role of the device is to preserve the corneal graft for transplant and facilitate the trifold, which unfurls easily within the eye of the patient. To the extent design changes known to or conceivable to one of skill in the art can be made while maintaining this objective, these changes are considered within the scope of this invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device comprising:
an inserter for holding a corneal graft, wherein the inserter is configured to hold the corneal graft in a tri-folded orientation, wherein the inserter comprises a distal end configured for insertion into the eye and a proximal end that is wider than the distal end;
a cap configured to couple to the inserter, wherein the cap is configured for covering the corneal graft in the inserter;
a handle that is configured to couple to the inserter for facilitating delivery of the corneal graft to an eye of a patient.

2. The device of claim 1 further comprising the inserter comprising a distal end with a flattened, ovular shape.

3. The device of claim 1 further comprising the cap having a front component and a back component that interlock.

4. The device of claim 1 further comprising the handle having a straight configuration.

5. The device of claim 1 further comprising the handle being bent at an angle to the inserter.

6. The device of claim 1 further comprising the handle being configured for facilitating a pull through delivery of the corneal graft to an eye of a patient.

7. The device of claim 1 further comprising the handle being configured for facilitating a fluid based delivery of the corneal graft to an eye of a patient.

8. The device of claim 1 further comprising the distal end of the inserter having a circular cross section and a bevel.

9. The device of claim 1 further comprising the proximal end of the inserter having an ovular cross-section and protrusions for attachment of modular interlocking handle pieces.

10. The device of claim 1 wherein the inserter defines a sufficient interior space and exterior protection to allow the graft to be stored in Optisol and in a trifold configuration.

11. The device of claim 1 wherein the inserter further comprises luminal axial troughs running from the proximal end halfway up a length of the inserter, wherein multiple troughs are arranged radially such that the corneal graft will lie in the tri-folded configuration with minimal luminal wall contact, and wherein arrangement and depth of the troughs also allows for fluid flow and facilitate grasping of graft by forceps.

12. The device of claim 1 further comprising the handle having the capability to aspirate and eject fluid.

13. The device of claim 1 further comprising the cap taking the form of a fluid-permeable cap.

14. The device of claim 1 further comprising the cap taking the form of a fluid-tight cap.

15. A method for Descemet's membrane endothelial keratoplasty (DMEK), comprising:
placing a corneal graft on an inserter for holding the corneal graft;
covering the corneal graft with a cap that is configured to couple to the inserter; and,
transmitting the corneal graft to the surgical center for performing the DMEK procedure.

16. The method of claim 15 further comprising storing the corneal graft in the inserter in a biocompatible fluid.

17. The method of claim 15 further comprising storing the corneal graft in the inserter in Optisol.

18. The method of claim 15 further comprising storing the corneal graft in a tri-fold configuration.

19. The method of claim 15 further comprising folding the graft in an opposite direction of a direction in which it naturally scrolls.

20. The method of claim 15 further comprising packaging the inserter for shipment.
